(19) United States
(12) Patent Application Publication
(10) Pub. No.: US 2019/0357772 A1
(43) Pub. Date: Nov. 28, 2019

(54) System and Method for In Situ Visualization of Nerves Using Targeted Flourescent Molecules

(71) Applicant: Avent, Inc., Alpharetta, GA (US)

(72) Inventors: Arne J. Madsen, Newport Beach, CA (US); Kenneth C. Hsu, Tustin, CA (US)

(21) Appl. No.: 16/331,997

(22) PCT Filed: Sep. 15, 2016
(86) PCT No.: PCT/US2016/051820

§ 371 Date: Mar. 11, 2019

(51) Int. Cl. A61B 5/00; A61B 5/055; A61B 6/03; A61B 8/08; A61B 6/12; A61K 49/00; A61M 25/06

(52) U.S. Cl. CPC A61B 5/0071; A61B 5/4821; A61B 5/055; A61B 6/032; A61B 5/4041; A61B 6/12; A61B 8/481; A61K 49/0097; A61M 25/0606; A61B 8/085

(57) ABSTRACT

The present invention is directed to a system and method for enhancing in situ visualization of a target site within a patient during a medical procedure using fluorescence. As such, the system includes a plurality of fluorescent molecules configured to selectively target and bind to one or more locations at the target site within the patient and a delivery mechanism for delivering the fluorescent molecules into the patient. In addition, the system includes a detection device configured to generate a detectable signal containing information relating to the target site and send the detectable signal to an imaging system for viewing by a user, such as a physician. Thus, the fluorescent molecules enhance in situ visualization of the target site when viewed through the skin of the patient.

### FIELD OF THE INVENTION

The present invention relates generally to the field of medical imaging, and more particularly, to a system and method for in situ visualization (i.e. through the skin) of nerves using targeted fluorescent molecules.

### BACKGROUND

Conventional imaging technologies such as CT, MRI, and ultrasound can be categorized as structural imaging modalities. Such imaging modalities are generally able to identify anatomical structures but are not without drawbacks. For example, certain imaging modalities are not particularly helpful during nerve block procedures, as the technologies have previously not been efficient at delivering clear images of the nerve block anatomy, surrounding structures, and/or the needle location.

Recent advancements in imaging modalities, however, have provided for effective nerve block procedures to be performed using such imaging. For example, in medical imaging, selective particles have been shown to be able to target certain cell types, such as cancer cells and/or nerve bundles. More specifically, magnetic materials and/or magnetic particles are often employed in the body to enhance image contrast of such cells. The magnetic nanoparticles can be passivated by biocompatible coatings such as dextrin, citrate, olystyrene, and/or divinylbenzene. These coatings can also detoxify the particles, resulting in enhanced lifetimes in vivo. Such targeted particles have shown promise in enhancing imaging of such cells using imaging modalities.

Thus, developments in structural imaging modalities that continuously improve upon medical imaging during various medical procedures, such as nerve block procedures, would be welcomed in the art. More specifically, a system and method for echogenically enhancing nerve fibers or bundles as well as enhancing in situ visualization (i.e. through the skin) using targeted fluorescent molecules would be advantageous.

### SUMMARY OF THE INVENTION

Objects and advantages of the invention will be set forth in part in the following description, or may be obvious from the description, or may be learned through practice of the invention.

In one aspect, the present invention is directed to a system for enhancing in situ visualization of a target site within a patient during a medical procedure. The system includes a plurality of fluorescent molecules configured to selectively target and bind to one or more locations at the target site within the patient. Further, the system includes a delivery mechanism for delivering the plurality of fluorescent molecules into the patient towards the target site. In addition, the system includes a detection device configured to generate a detectable signal corresponding to the target site and send the detectable signal to an imaging system. Thus, the fluorescent molecules enhance in situ visualization of the target site when viewed through the skin of the patient as well as when viewed via the imaging system.

In one embodiment, the detection device may include at least one sensor having an emitter and a receiver. More specifically, in certain embodiments, the sensor(s) may include a fiber optic sensor.

In another embodiment, each of the plurality of fluorescent molecules may include at least one of a fluorescent moiety, a fluorescent protein, a peptide, or a fluorescent dye. In additional embodiments, each of the fluorescent molecules may have a diameter of from about 1 nanometer to about 100 nanometers.

In further embodiments, the fluorescent molecules may be suspended in a liquid medium in a quantity of from about one thousand (1,000) to about one million (1,000,000) fluorescent molecules. In such embodiments, the liquid medium may be delivered, via the delivery mechanism, to the target site via a plurality of phages. For example, in one embodiment, the delivery mechanism may include a needle and/or a syringe.

In certain embodiments, the plurality of fluorescent molecules may temporarily bind to the one or more locations at the target site for a predetermined dwell time before diffusing into the patient. In such embodiments, the predetermined dwell time of the plurality of fluorescent molecules may include from about one day to about two days.

In yet another embodiment, the target site of the patient may include nerve cells, cancer cells, nerve sheaths, nerve bundles, nerve fibers, or any other nerves and/or cells within the patient. For example, in one embodiment, the medical procedure may include a peripheral nerve block procedure.

In additional embodiments, the imaging system may include a CT scanner, an MRI scanner, an ultrasound imaging system, or similar. More specifically, in certain embodiments, the imaging system may include, at a minimum, a display for viewing the target site. As such, the fluorescent molecules are configured to echogenically enhance the target site when viewed by the display.

In another aspect, the present invention is directed to a method for detecting a target site within a patient through the patient's skin during a medical procedure. The method includes delivering, via a delivery mechanism, a plurality of fluorescent molecules into the patient towards the target site. The method also includes allowing the plurality of fluorescent molecules to selectively target and bind to the target site. Once the fluorescent molecules have bound to the target site, the method also includes viewing the target site of the patient through the patient's skin.

In one embodiment, the step of delivering the plurality of fluorescent molecules into the patient towards the target site may include suspending the plurality of fluorescent molecules in a liquid medium and delivering the liquid medium to the target site via a plurality of phages.

In another embodiment, the method may also include adjusting a quantity of the fluorescent molecules being delivered into the patient as a function of the medical procedure.

In further embodiments, the method may include viewing the target site of the patient through the patient's skin via a detection device that generates a detectable signal containing information related to the target site and sends the detectable signal to an imaging system. More specifically, in certain embodiments, the detection device may include at least one sensor having an emitter and a receiver. For example, as mentioned, the sensor(s) may include a fiber optic sensor. It should be understood that the method may further include any of the additional method steps/or features as described herein.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a schematic representation of one embodiment of a system for enhancing in situ visualization of a target site within a patient during a medical procedure using fluorescence according to the present disclosure;

FIG. 2 illustrates various examples of optical fiber designs of fiber optic sensors according to the present disclosure;

FIG. 3 illustrates schematic representations of various optical fiber bundles of fiber optic sensors according to the present disclosure;

FIG. 4 illustrates a schematic representation of a portion of a detection device having a sensor according to the present disclosure;

FIG. 5 illustrates a schematic diagram of one embodiment of an imaging system according to the present disclosure;

FIG. 6 illustrates a schematic diagram of one embodiment of suitable components that may be included in a processor of the imaging system of FIG. 5;

FIG. 7 illustrates a schematic diagram of one embodiment of a probe configured on a patient's skin so as to generate an image of a target site of the patient according to the present disclosure;

FIG. 8 illustrates a schematic diagram of one embodiment of a probe configured on a patient's skin so as to generate an image of a target site of a patient according to the present disclosure, particularly illustrating a delivery mechanism delivering a plurality of targeted particles into the patient towards the target site; and

FIG. 9 illustrates a flow diagram of one embodiment of a method for detecting a target site within a patient through the patient's skin during a medical procedure according to the present disclosure.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

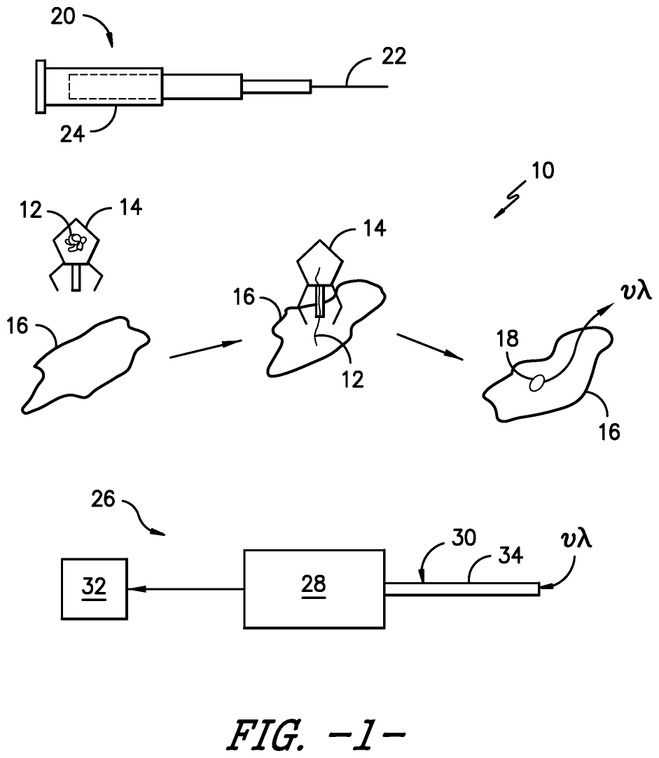

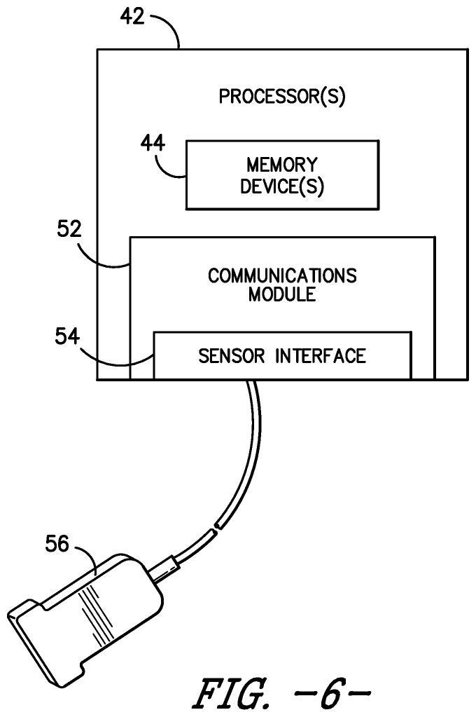

Referring now to the drawings, FIG. 1 illustrates a system 10 for enhancing in situ visualization of a target site 16 within a patient during a medical procedure using fluorescence. In certain embodiments, the target site 16 of the patient may include nerve cells, cancer cells, nerve sheaths, nerve bundles, nerve fibers, or any other nerves and/or cells within the patient. Thus, in one embodiment, the medical procedure may include a peripheral nerve block procedure. More particularly, as shown, the system 10 includes a plurality of fluorescent molecules 12 configured to selectively target and bind to one or more locations at the target site 16 so as to encode a detectable marker 18 within the patient. Further, the system 10 includes a delivery mechanism 20 for delivering the fluorescent molecules 12 into the patient. In addition, the system 10 may include a detection device 26 configured to generate and send a detectable signal containing information relating to the target site 16 to an imaging system 10 (FIG. 5) for viewing by a user, such as a physician. Thus, the fluorescent molecules 12 enhance in situ visualization of the target site when viewed through the skin of the patient as well as via an imaging system.

More specifically, the fluorescent molecules 12 may be suspended in a liquid medium in a quantity of from about one thousand (1,000) to about one million (1,000,000) fluorescent molecules. Thus, as shown, the delivery mechanism 26 is configured to deliver the liquid medium that contains the fluorescent molecules 12 into the patient via one or more phages 14 that may specifically bind to the target site 16. The delivery mechanism 20 as described herein may include a syringe 24 configured with a needle 22, a needle-guide assembly, or any other suitable delivery mechanism. Further, in certain embodiments, where the delivery mechanism 20 corresponds to a needle guide assembly, the assembly may include, at least, a needle and a catheter. As such, it should be understood that the needle as well as the catheter of the needle guide assembly can be inserted into the patient in any particular order or simultaneously so as to deliver the fluorescent molecules 12 described herein. For example, in one embodiment, the needle guide assembly may include an over-the-needle (OTN) catheter assembly in which the catheter is coaxially mounted over the needle. Alternatively, the needle may be mounted over the catheter. In such embodiments, the needle may act as an introducer such that it places the catheter within the patient to deliver the fluorescent molecules 12 and is later removed.

The fluorescent molecules 12 as described herein may include any suitable fluorescence including but not limited to a fluorescent moiety, a fluorescent protein, a peptide, or a fluorescent dye. For example, in certain embodiments, pharmaceutical compositions may include the targeting fluorescent molecules 12 disclosed herein. Pharmaceutical compositions as described herein may be formulated using one or more physiologically acceptable carriers including excipients and auxiliaries which facilitate processing of the active agents into preparations which are used pharmaceutically. Proper formulation is dependent upon the route of administration chosen.

In certain embodiments, the pharmaceutical composition disclosed herein may include a pharmaceutically acceptable diluent(s), excipient(s), or carrier(s). In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

In certain embodiments, the pharmaceutical compositions disclosed herein may be administered to a patient by any suitable administration route, including but not limited to, parenteral (intravenous, subcutaneous, intraperitoneal, intramuscular, intravascular, intrathecal, intravitreal, infusion, or local) administration. More specifically, formulations that are suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent may be optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein may be in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions may be optionally prepared as appropriate oily injection suspensions.

In some embodiments, the pharmaceutical composition described herein may be in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

In addition, as mentioned, the fluorescent molecules 12 may include a fluorescent moiety (e.g., a fluorescent protein, peptide, or fluorescent dye molecule). As such, all fluorescent moieties are encompassed within the term “fluorescent moiety.” Specific examples of fluorescent moieties given herein are illustrative and are not meant to limit the fluorescent moieties for use with the targeting molecules disclosed herein.

Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes.

In some embodiments, the fluorescent moiety may be a fluorescein dye. Examples of fluorescein dyes include, but are not limited to, 5-carboxyfluorescein, fluorescein-5-isothiocyanate and 6-carboxyfluorescein. In further embodiments, the fluorescent moiety may be a rhodamine dye. Examples of rhodamine dyes include, but are not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). In additional embodiments, the fluorescent moiety may be a cyanine dye. Examples of cyanine dyes include, but are not limited to, Cy3, Cy3B, Cy3.5, Cy5, Cy5.5, Cy7. In particular embodiments, the fluorescent moiety may be a peptide. In some embodiments, the fluorescent moiety is Green Fluorescent Protein (GFP). In some embodiments, the fluorescent moiety is a derivative of GFP (e.g., EBFP, EBFP2, Azurite, mKalamal, ECFP, Cerulean, CyPet, YFP, Citrine, Venus, YPet).

In some embodiments, the fluorescent moiety may be conjugated to high molecular weight molecule, such as water soluble polymers including, but not limited to, dextran, PEG, serum albumin, or poly(amidoamine) dendrimer.

In addition, in certain embodiments, a cargo (e.g., a drug) may be directly attached to the fluorescent molecules 12. Alternatively, a cargo be indirectly attached to a targeting molecule disclosed herein (e.g., via a linker). As used herein, a “linker” generally refers to any molecule capable of binding (e.g., covalently) to a targeting molecule disclosed herein. Linkers include, but are not limited to, straight or branched-chain carbon linkers, heterocyclic carbon linkers, peptide linkers, and polyether linkers. In some embodiments, the linker binds to the fluorescent molecules 12 disclosed herein by a covalent linkage. For example, in certain embodiments, the covalent linkage includes an ether bond, thioether bond, amine bond, amide bond, carbon-carbon bond, carbon-nitrogen bond, carbon-oxygen bond, or carbon-sulfur bond.

More specifically, the fluorescent molecules 12 may be selected based on their chemical or atomic structure being attracted to one or more locations at the target site 16. For example, the target site 16 of the patient may include a nerve bundle having a plurality of nerve fibers. Thus, the fluorescent molecules 12 are configured to selectively target and bind to one or more of the nerve fibers during a nerve block procedure. In such embodiments, when the fluorescent molecules 12 are injected into the patient, the fluorescent molecules 12 are attracted to the nerve fibers at the target site 16 and will easily bind thereto or form bonds therewith.

Determining whether the fluorescent molecules 12 are capable of binding to a neuron or nerve or component thereof is accomplished by any suitable method. For example, in some embodiments, determining whether the fluorescent molecules 12 are capable of binding to a neuron or nerve or component thereof may include contacting one of the fluorescent molecules 12 with a test agent for a period of time sufficient to allow the targeting molecule and test agent to form a binding complex. The binding complex may be detected using any suitable method. For example, in particular embodiments, suitable binding assays can be performed in vitro or in vivo and include, but are not limited to, phage display, two-hybrid screens, co-precipitation, cross-linking, and expression cloning. Other binding assays involve the use of mass spectrometry or NMR techniques to identify molecules bound to the target of interest. The targeting molecule utilized in such assays can be naturally expressed, cloned or synthesized.

Further, it should be understood that the plurality of fluorescent molecules 12 temporarily bind to the one or more locations at the target site 16 for a predetermined dwell time before diffusing into the patient as the human body naturally tries to remove any foreign materials that are present therein. As such, in certain embodiments, the predetermined dwell time of the fluorescent molecules 12 may include from about one day to about two days. In further embodiments, any suitable dwell time may be sufficient for binding the fluorescent molecules 12 to the target site 16 and then diffusing into the body, including less than one day or more than two days. For example, in one embodiment, the dwell time may correspond to a predetermined number of hours substantially corresponding to the length of the medical procedure.

In additional embodiments, the fluorescent molecules 12 as described herein may have any suitable size. For example, in certain embodiments, the fluorescent molecules 12 may correspond to nanoparticles. As used herein, the term ‘nanoparticles’ generally refers to extremely small particles that have a diameter of from about 1 nanometer to about 100 nanometers.

It should be understood that any suitable quantity of the fluorescent molecules 12 may be injected into the patient. For example, as mentioned, from about one thousand (1,000) to about one million (1,000,000) of the fluorescent molecules 12 may be injected or delivered into the patient and can be determined based on the procedure and/or the anatomical structure or surrounding tissue of the target site 16. In additional embodiments, any number of fluorescent molecules 12 may delivered into the patient, including less than 1,000 particles or more than 1,000,000 particles, e.g. depending on the medical procedure and/or the properties of the target site 16.

Following initial binding, the fluorescent molecules 12 may be inserted into and taken up by the target site 16 upon which the fluorescent molecules 12 may transcribe and produce the detectable marker 18. The optical signal produced by the detectable marker 18 (vA) may be detected, e.g. as with the detection device 26 described at more length below, and appropriate visualization may be obtained therefrom. More specifically, as mentioned, the detection device 26 is configured to generate and send the detectable signal 18 (vA) of the target site 16 to an imaging system (FIG. 5) for viewing by a user, such as a physician. Thus, the fluorescent molecules 12 enhance in situ visualization of the target site 16 when viewed through the skin of the patient. Fluorescent labels may be detected by any suitable method. For example, a fluorescent label may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultipliers, etc. More specifically, as shown, the fluorescent particles 12 may be detected via the detection device 26 that may include at least one sensor 28 having an emitter 30 and a receiver 32 that may further include a light source (not shown).

Referring now to FIGS. 1-3, upon generation of the detectable signal 18 (vA), the sensor(s) 28 of the detection device 26 may be utilized to detect and transmit the detectable signal 18 to appropriate personnel. For example, in one embodiment, disclosed methods may utilize a fiber optic-based sensor, one or more fiber optic cables of which may be located at the target site 16. Optionally, a fiber optic cable of the sensor(s) 28 may carry a delivery vehicle that in turn may carry the fluorescent molecules 12 as described herein, e.g. the liquid medium containing the phages 14. Beneficially, optical fibers may be formed of biocompatible materials that may remain at a site of interest for a relatively long period of time, for instance to monitor the site during a medical procedure. In addition, at the time of removal, optical fibers may be easily removed from the site without the necessity of causing excessive tissue damage at the site, due to the small cross-section of the fibers.

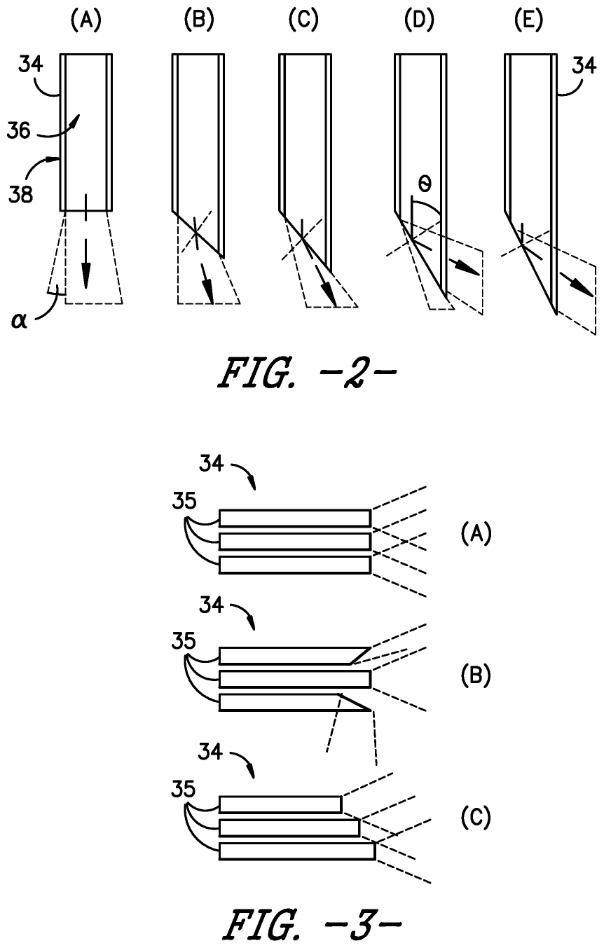

Referring particularly to FIG. 2, schematic diagrams of several embodiments of optical fibers that may be utilized in a sensor according to certain disclosed detection methods are illustrated. For example, as shown, an optical fiber may include a core 36, through which light may travel, and an external cladding layer 38. The difference in the index of refraction between the core 36 and the cladding layer 38 defines the critical angle at which total internal reflection takes place at the core/clad interface. Thus, light that impinges upon the interface at an angle greater than the critical angle is completely reflected, allowing the light to propagate down the fiber.

Optical fibers may generally include multi-mode fibers having a core diameter greater than about 10 micrometers (μm). The preferred core diameter in any particular embodiment may depend upon the characteristics of excitation light (when required) and/or emission light, among other system parameters. For instance, in those embodiments in which a laser is the excitation source, a core diameter may be between about 50 μm and about 100 μm, or about 80 μm. In other embodiments, for instance, in which an excitation light source produces less coherent radiation, such as a multi-wavelength light emitting diode (LED), for example, it may be preferable to utilize an optical fiber having a larger core diameter, for instance between about 90 μm and about 400 μm.

The core/clad boundary of the cables 34 may be abrupt, as in a step-index fiber, or may be gradual, as in a graded-index fiber. A graded-index fiber may be preferred in some embodiments, as graded index fibers may reduce dispersion of multiple modes traveling through the fiber. This is not a requirement of disclosed sensors, however, and step-index fibers may alternatively be utilized, particularly in those embodiments in which the optical fiber is of a length such that dispersion will not be of great concern.

Optical fibers may be formed of sterilizable, biocompatible materials that may be safely placed and held at a potential target site, and in one particular embodiment, at a surgical site. For example, optical fibers formed of any suitable type of glass may be used, including, without limitation, silica glass, fluorozirconate glass, fluoroaluminate glass, any chalcogenide glass, or the like may form the core and/or the clad. Polymer optical fibers (POF) are also encompassed by the present disclosure. For instance, optical fibers formed of suitable acrylate core/clad combinations, e.g., polymethyl methacrylates, may be utilized. It may be preferred in some embodiments to utilize a multi-core POF so as to lower losses common to POF due to bending of the fiber. For instance, this may be preferred in those embodiments in which the optical fiber(s) of the sensor are in a non-linear conformation during use.

The end of the fiber may be shaped as desired. For instance, and as illustrated in FIGS. 2(A)-2(E), polishing or otherwise forming a specific angle at the end face of a fiber may maintain the acceptance angle α and collection efficiency of the fiber, while rotating the field of view of the fiber, as depicted by the arrows. Depending upon the angle at the fiber end, light may enter the fiber from angles up to about 90° of the fiber axis (e.g., as shown at FIG. 2(E).

Optical fibers of a sensor may be formed so as to detect light at locations along the length of the fiber, in addition to at the terminal end of the fiber. For instance, at locations along the length of the fiber may be bent or notched so as to allow light through the cladding layer 38, optionally at a predetermined angle, such that excitation light (when needed) may enter the optical fiber at these locations. For example, the cladding layer 38 of a fiber may be bent or otherwise notched at a predetermined angle to form a ‘window’ in the fiber.

A fiber optic sensor for use as described herein may further include a fiber optic cable comprised of a single optical fiber or a plurality of optical fibers, depending upon the specific design of the sensor. For instance, a plurality of optical fibers may be joined to form a single fiber cable of a size to be located at an in vivo site of interest (e.g., less than about 1.5 mm in cross-sectional diameter).

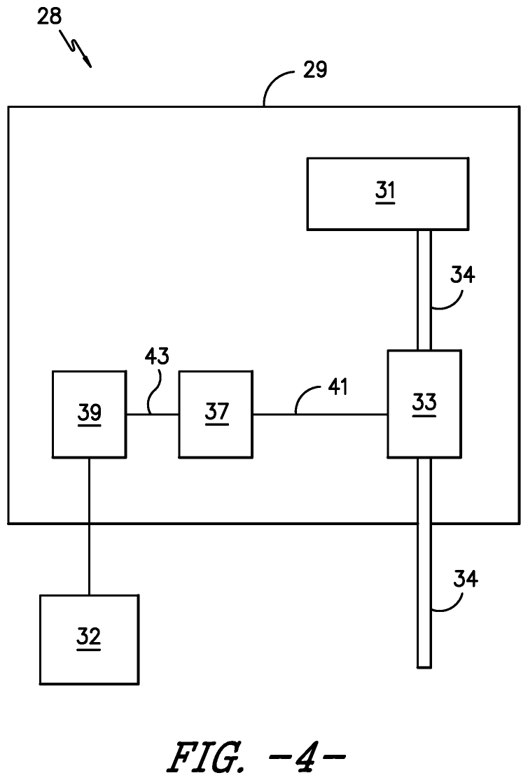

When utilizing a plurality of fibers in a fiber bundle or cable, individual fibers may be formed and arranged in relation to one another so as to provide a wider angle of detection. For instance, FIGS. 3(A)-3(C) illustrate several different embodiments of a fiber optic cable 34 having multiple optical fibers 35 in a bundle. More specifically, as shown at FIG. 3(A), through location of a plurality of fiber ends at a single cross-sectional area, improved light collection may be attained, as the total field area covered by the combined fibers 35 will be larger than that for a single fiber 35. Further, as shown in the illustrated embodiment of FIG. 3(B), the geometry of the end face of different fibers 35 contained in the cable 34 may be different from one another, so as to allow light collection from a variety of different directions. In addition, as shown in FIG. 3(C), the fiber ends may be staggered over a length, so as to increase the axial length of the light collection area and increase the area of inquiry in an axial direction. Of course, combinations of such designs, as well as other fiber design for improving the collection of a signal area, including methods as discussed above as well as methods as are generally known to those in the art, may be utilized as well.

A fiber optic bundle or cable 34 of optical fibers 35 may generally be held as a cohesive unit with any biocompatible sheath that can hold the unit together while maintaining flexibility of the fibers 35. For instance, a fiber optic cable 34 may include an outer sheath of a flexible polyurethane.

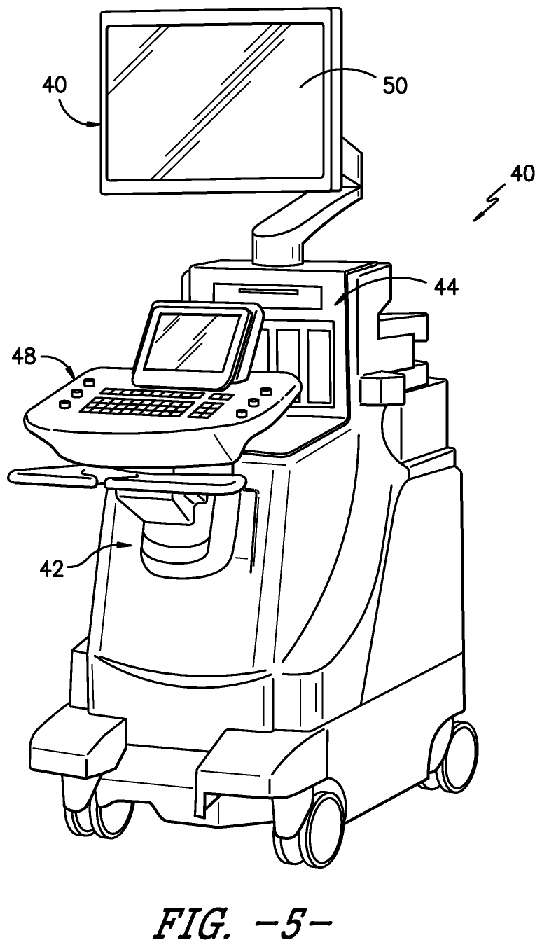

In accordance with the present technology, one or more optical fibers 35 may be utilized as a portion of the sensor 28 that can be contained by use of a portable device, one embodiment of which is schematically illustrated in FIG. 4. More specifically, as shown, the sensor 28 includes several components that may be housed within an enclosure 29. For example, the enclosure 29 may be, for example, a molded plastic enclosure of a size so as to be easily held by a physician. For instance, the enclosure 29 may include clips, loops, or the like so as to be attachable to a physician's clothing or body. In general, the enclosure 29 may be relatively small, for instance less than about 10 cm by about 8 cm by about 5 cm, so as to be inconspicuously carried by a physician and so as to avoid impedance of a physician's motion. Further, the enclosure 29 may completely enclose the components contained therein, or may partially enclose the components contained therein. For example, the enclosure 29 may include an access port (not shown) that may provide access to the interior of enclosure 29. In one embodiment, an access port may be covered with a removable cover, as is known in the art.

Still referring to FIG. 4, the enclosure 29 may house a power supply 31 that may be configured to supply power to the various operational components housed therein. In one embodiment, the power supply 31 may correspond to a battery, however, those of ordinary skill in the art will appreciate that other power supplies may be used including those that may be coupled to an external alternating current (AC) supply so that the enclosed power supply may include those components necessary to convert such external supply to a suitable source for the remaining components requiring a power source.

Further, as shown, the fiber optic cable 34 is configured to extend externally from the enclosure 29 to the field of inquiry, e.g., within the target site 16. In addition, the enclosure 29 may further house an optical detector 33 coupled to the fiber optic cable 34. The optical detector 33 may correspond to a photodiode, a photoresistor, or the like. Further, the optical detector 33 may include optical filters, beam splitters, and so forth that may remove background light and reduce the total input optical signal at the optical detector 33 to one or more diagnostically relevant emission peaks. Moreover, the optical detector 33 may produce a signal proportional to targeted emission peaks and couple such signal to line 41 for transmission to signal processor 37.

The signal processor 37 may include a microprocessor configured to evaluate the strength or other characteristics of the output signal received over line 41 to, e.g., correlate the optical signal to the fluorescent molecules 12 at the target site 16 and to produce a detection signal that may be coupled to line 43 for passage to a signaling device 39. Accordingly, a detectable signal may be initiated at signaling device 39. In an exemplary configuration, a detectable signal may initiate a visible or audible signal within or at the surface of the enclosure 29 by way of signaling device 39 that may be detected by the wearer. In addition to or alternative to a visual and/or audible signal at the enclosure 29 itself, the signaling device 39 may include a transmitter portion that, upon initiation of the detectable signal, may transmit an electromagnetic signal to the receiver 32. Further, as shown, the receiver 32 may be remote from the signaling device 39. For instance, the receiver 32 may be on the wearer's body at a distance from the signaling device 39, at a location apart from the wearer's body that may be conveniently chosen by the wearer.

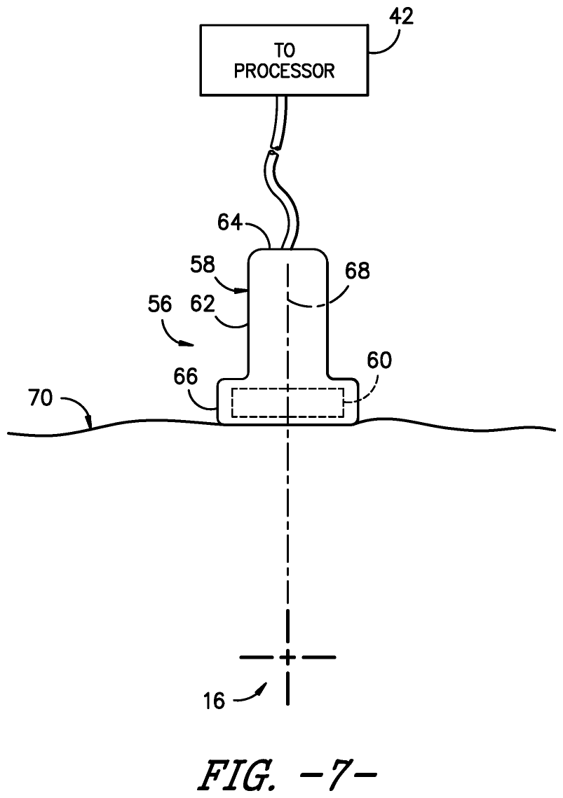

As mentioned, the detection device 26 described herein may also be used in conjunction with an imaging system 40 (FIG. 5). More specifically, in certain embodiments, the detection device 26 is configured generate and send the detectable signal vλ from the target site 16 to the imaging system 40. For example, as shown in FIGS. 5 and 6, one embodiment of an imaging system 40 and an associated processor 42 thereof according to the present disclosure are illustrated, respectively. As described herein, the imaging system 40 may correspond to an ultrasound imaging system (as shown), a computer tomography (CT) scanner, a magnetic resonance imaging (MRI) scanner, or any other suitable imaging system. Further, as shown, the imaging system 40 generally includes one or more processor(s) 42 and associated memory device(s) 44 configured to perform a variety of computer-implemented functions (e.g., performing the methods and the like and storing relevant data as disclosed herein), as well as a user display 46. In addition, the imaging system 40 may include a user interface 48, such as a computer and/or keyboard, configured to assist a user in generating and/or manipulating an image 50 displayed by the user display 46. As such, the fluorescent molecules 12 are configured to echogenically enhance the target site 16 when viewed by the display 46.

Additionally, as shown in FIG. 6, the processor(s) 42 may also include a communications module 52 to facilitate communications between the processor(s) 42 and the various components of the imaging system 40, e.g. any of the components of FIG. 2. Further, the communications module 52 may include a sensor interface 54 (e.g., one or more analog-to-digital converters) to permit signals transmitted from one or more probes (e.g. the ultrasound probe 56) to be converted into signals that can be understood and processed by the processor(s) 42. It should be appreciated that the ultrasound probe 56 may be communicatively coupled to the communications module 52 using any suitable means. For example, as shown in FIG. 6, the ultrasound probe 56 may be coupled to the sensor interface 54 via a wired connection. However, in other embodiments, the ultrasound probe 56 may be coupled to the sensor interface 54 via a wireless connection, such as by using any suitable wireless communications protocol known in the art. As such, the processor(s) 42 may be configured to receive one or more signals from the ultrasound probe 56.

As used herein, the term “processor” refers not only to integrated circuits referred to in the art as being included in a computer, but also refers to a controller, a microcontroller, a microcomputer, a programmable logic controller (PLC), an application specific integrated circuit, a field-programmable gate array (FPGA), and other programmable circuits. The processor(s) 42 is also configured to compute advanced control algorithms and communicate to a variety of Ethernet or serial-based protocols (Modbus, OPC, CAN, etc.). Furthermore, in certain embodiments, the processor(s) 42 may communicate with a server through the Internet for cloud computing in order to reduce the computation time and burden on the local device. Additionally, the memory device(s) 44 may generally comprise memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) 44 may generally be configured to store suitable computer-readable instructions that, when implemented by the processor(s) 42, configure the processor(s) 42 to perform the various functions as described herein.

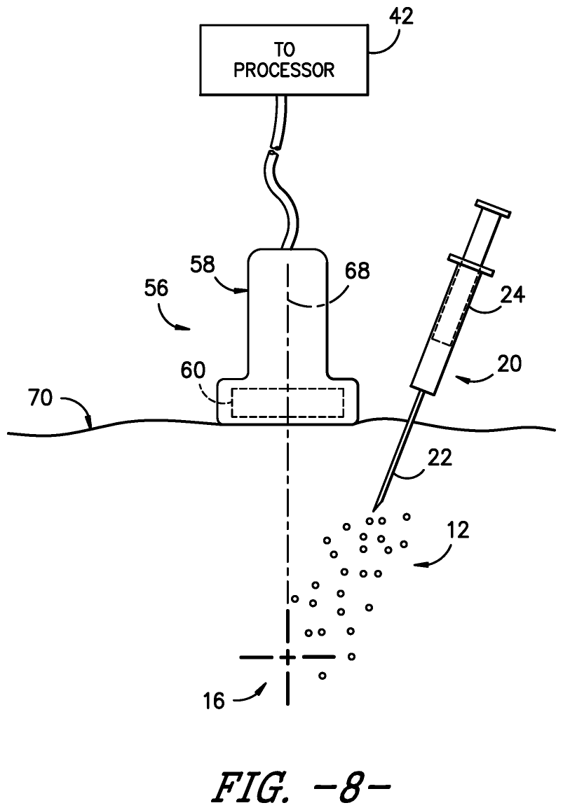

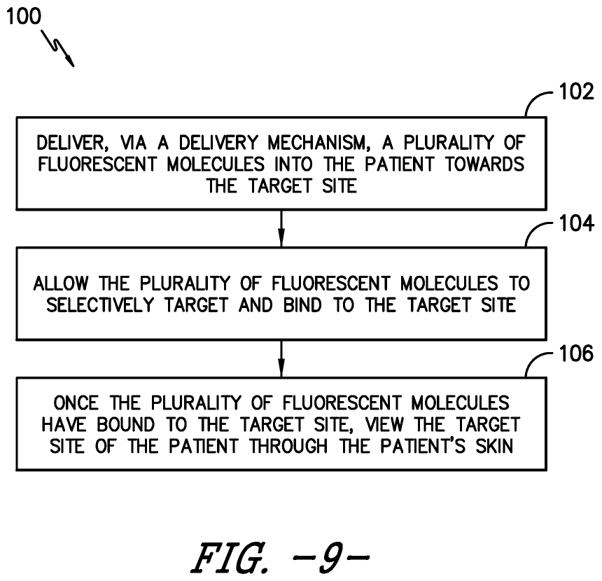

Thus, as shown in FIGS. 7 and 8, the ultrasound probe 56 may include a transducer housing 30 and a transducer transmitter 60 mounted therein. As is generally understood, the transducer transmitter 60 is configured to emit and/or receive ultrasound beams. For example, as shown in FIG. 7, the transducer housing 58 includes a body 62 extending from a proximal end 64 to a distal end 66 along a longitudinal axis 68 that runs along the length of the body 62. Further, the distal end 66 of the body 62 includes an internal cavity (not numbered). As such, the transducer transmitter 60 may be configured within the internal cavity so as to scan the target site 16 within a patient when the ultrasound probe 56 is placed on the patient's skin 70 during a medical procedure. An image 50 of the target site 16 can then be generated and displayed to a user via the display 46 of the ultrasound imaging system 10 (FIG. 5).

Referring now to FIG. 9, a flow diagram of one embodiment of a method 100 for detecting a target site within a patient through the patient's skin during a medical procedure is illustrated. As shown at 102, the method 100 includes delivering, via the delivery mechanism 20, a plurality of fluorescent molecules 12 into the patient towards the target site 16. More specifically, in one embodiment, the step of delivering the fluorescent molecules 12 into the patient towards the target site 16 may include suspending the fluorescent molecules 12 in a liquid medium and delivering the liquid medium to the target site 16 via a plurality of phages 14.

In addition, as shown at 104, the method 100 includes allowing the plurality of fluorescent molecules 12 to selectively target and bind to the target site 16. Once the fluorescent molecules have bound to the target site, as shown at 106, the method 100 includes viewing the target site 16 of the patient through the patient's skin. In another embodiment, the method 100 may also include adjusting a quantity of the fluorescent molecules 12 being delivered into the patient as a function of the medical procedure.

In further embodiments, the method 100 may include viewing the target site 16 of the patient through the patient's skin via a detection device 26 that generates a detectable signal containing information related to the target site 16. Further, the detection device 26 is configured to send the detectable signal to an imaging system, e.g. such as the imaging system 10 of FIG. 5.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

1. A system for enhancing in situ visualization of a target site within a patient during a medical procedure, the system comprising:
a plurality of fluorescent molecules configured to selectively target and bind to one or more locations at the target site within the patient;
a delivery mechanism for delivering the plurality of fluorescent molecules into the patient towards the target site; and,
a detection device comprising a light source, the detection device configured to:
generate a detectable signal comprising information relating to the target site, and
send the detectable signal to an imaging system, wherein the light source illuminate the fluorescent molecules so as to enhance in situ visualization of the target site when viewed through the skin of the patient.

2. The system of claim 1, wherein the detection device further comprises at least one sensor comprising an emitter and a receiver.

3. The system of claim 2, wherein the sensor comprises a fiber optic sensor.

4. The system of claim 1, wherein each of the fluorescent molecules comprises a diameter of from about 1 nanometer to about 100 nanometers.

5. The system of claim 1, wherein the fluorescent molecules are suspended in a liquid medium in a quantity of from about one thousand (1,000) to about one million (1,000,000) fluorescent molecules.

6. The system of claim 5, wherein the liquid medium is delivered, via the delivery mechanism, to the target site via a plurality of phages.

7. The system of claim 1, wherein the delivery mechanism comprises at least one of a needle or a syringe.

8. The system of claim 1, wherein each of the plurality of fluorescent molecules comprises at least one of a fluorescent moiety, a fluorescent protein, a peptide, or a fluorescent dye.

9. The system of claim 1, wherein the plurality of fluorescent molecules temporarily bind to the one or more locations at the target site for a predetermined dwell time before diffusing into the patient, wherein the predetermined dwell time of the plurality of fluorescent molecules comprises from about one day to about two days.

10. The system of claim 1, wherein the target site of the patient comprises at least one of nerve cells, cancer cells, nerve sheaths, nerve bundles, or nerve fibers.

11. The system of claim 1, wherein the medical procedure comprises a peripheral nerve block procedure.

12. The system of claim 1, wherein the imaging system comprises at least one of a CT scanner, an MRI scanner, or an ultrasound imaging system, the imaging system comprising a display for viewing the target site, wherein the fluorescent molecules echogenically enhance the target site when viewed by the display.

13. A method for detecting a target site within a patient through the patient's skin during a medical procedure, the method comprising:
delivering, via a delivery mechanism, a plurality of fluorescent molecules into the patient towards the target site;
allowing the plurality of fluorescent molecules to selectively target and bind to the target site; and,
once the plurality of fluorescent molecules have bound to the target site, viewing the target site of the patient through the patient's skin.

14. The method of claim 13, wherein delivering the plurality of fluorescent molecules into the patient towards the target site further comprises suspending the plurality of fluorescent molecules in a liquid medium and delivering the liquid medium to the target site via a plurality of phages.

15. The method of claim 13, further comprising adjusting a quantity of the fluorescent molecules being delivered into the patient as a function of the medical procedure.

16. The method of claim 13, further comprising viewing the target site of the patient through the patient's skin via a detection device that generates a detectable signal containing information related to the target site and sends the detectable signal to an imaging system.

17. The method of claim 16, wherein the detection device further comprises at least one sensor comprising an emitter and a receiver, and wherein the sensor comprises a fiber optic sensor.

18. The method of claim 13, wherein each of the plurality of fluorescent molecules comprises at least one of a fluorescent moiety, a fluorescent protein, a peptide, or a fluorescent dye.

19. The method of claim 13, wherein the target site of the patient comprises at least one of nerve cells, cancer cells, nerve sheaths, nerve bundles, or nerve fibers, and wherein the medical procedure comprises a peripheral nerve block procedure.

20. The method of claim 13, wherein the imaging system comprises at least one of a CT scanner, an MRI scanner, or an ultrasound imaging system, the imaging system comprising a display for viewing the target site, wherein the fluorescent molecules echogenically enhance the target site when viewed by the display.
